(19) United States
(12) Patent Application Publication
(10) Pub. No.: US 2011/0106200 A1
(43) Pub. Date: May 5, 2011

(54) STROKE RISK MONITORING SYSTEM INCLUDING IMPLANTABLE MEDICAL DEVICE

(75) Inventor: Paul D. Ziegler, Minneapolis, MN (US)

(73) Assignee: Medtronic, Inc., Minneapolis, MN (US)

(21) Appl. No.: 12/608,889

(22) Filed: Oct. 29, 2009

(51) Int. Cl. A61N 1/365; A61B 5/0402

(52) U.S. Cl. 607/18; 600/513

(57) ABSTRACT

One or more example techniques for monitoring the stroke risk of a patient via a system including an implantable medical device. In some examples, a method including monitoring at least one physiological parameter of a patient via an implantable medical device; determining whether each of a plurality stroke risk factors are present based at least in part on the at least one physiological parameters monitored via the implantable medical device; and generating a stroke risk score based on the stroke risk factors determined to be present, wherein the stroke risk score is reflective of the patient's risk of stroke.

### TECHNICAL FIELD

The present disclosure relates to implantable medical devices and, more particularly implantable medical devices for monitoring stroke risk in a patient.

### BACKGROUND

Stroke is a serious medical condition that can cause permanent neurological damage, complications, and death. Stroke may be characterized as the rapidly developing loss of brain functions due to a disturbance in the blood vessels supplying blood to the brain. The loss of brain functions can be a result of ischemia (lack of blood supply) caused by thrombosis or embolism. During a stroke, the blood supply to an area of a brain may be decreased, which can lead to dysfunction of the brain tissue in that area.

A variety of approaches exist for treating patients with a high risk of stroke. For example, anticoagulants, such as warfarin, can be effective in reducing the risk of ischemic stroke. However, such anticoagulants may be frequently underprescribed due to the failure to timely identify the presence of one or more patient risk factors that correlate to a relatively high risk of stroke.

### SUMMARY

In general, the disclosure is directed to systems and techniques for assessing a patient's stroke risk based on one or more physiological parameters of the patient monitored via an implantable medical device (IMD). A patient's stroke risk may be reflected by a stroke risk score generated by a stroke risk monitoring system including the IMD. The risk monitoring system may generate a patient stroke risk score based on patient stroke risk factors, such as, e.g., atrial fibrillation, hypertension, congestive heart failure, diabetes, and the like, identified by the monitoring system as being present in a patient. The risk monitoring system may detect the presence of one or more stroke risk factors used to compute a stroke risk score of the patient based on one or more physiological parameters of the patient monitored via the implantable medical device. The risk monitoring system may monitor for the presence of a plurality of stroke risk factors used to determine the stroke risk score a patient to track a patient's risk of stroke. In some examples, the stroke risk monitoring system may alert the patient or a clinician based on the stroke risk score determined by the monitoring system, e.g., when the patient stroke risk score indicates an elevated stroke risk for the patient.

In one aspect, the disclosure is directed to a method comprising monitoring at least one physiological parameter of a patient via an implantable medical device; determining whether each of a plurality stroke risk factors are present based at least in part on the at least one physiological parameters monitored via the implantable medical device; and generating a stroke risk score based on the stroke risk factors determined to be present, wherein the stroke risk score is reflective of the patient's risk of stroke.

In another aspect, the disclosure is directed to stroke risk monitoring system comprising an implantable medical device including a sensing module configured to monitor at least one physiological parameter of a patient; and a processor configured to determine whether each of a plurality of stroke risk factors are present based at least in part the at least one physiological parameter, and generate a stroke risk score based on the stroke risk factors determined to be present, wherein the stroke risk score is reflective of the patient's risk of stroke.

In another aspect, the disclosure is directed to a system comprising means for monitoring at least one physiological parameter of a patient via an implantable medical device; means for determining whether each of a plurality stroke risk factors are present based at least in part on the at least one physiological parameters monitored via the implantable medical device; and means for generating a stroke risk score based on the stroke risk factors determined to be present, wherein the stroke risk score is reflective of the patient's risk of stroke.

In another aspect, the disclosure is directed to a computer-readable storage medium comprising instructions that cause a processor to monitor at least one physiological parameter of a patient via an implantable medical device; determine whether each of a plurality stroke risk factors are present based at least in part on the at least one physiological parameters monitored via the implantable medical device; and generate a stroke risk score based on the stroke risk factors determined to be present, wherein the stroke risk score is reflective of the patient's risk of stroke.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example stroke monitoring system.

FIG. 2 is a conceptual diagram illustrating an example implantable medical device.

FIG. 3 is a functional block diagram of an example implantable medical device that monitors the stroke risk of a patient.

FIG. 4 is a functional block diagram illustrating an example sensing module of the implantable medical device of FIG. 3.

FIG. 5 is a functional block diagram of an example medical device programmer.

FIG. 6 is a flow diagram illustrating an example technique for monitoring the stroke risk of a patient using an implantable medical device.

FIG. 7 is a block diagram illustrating an example system that includes an external device, such as a server, and one or more computing devices that are coupled to the IMD and programmer shown in FIG. 1 via a network.

### DETAILED DESCRIPTION

In general, the disclosure is directed to systems and techniques for assessing a patient's stroke risk based on one or more physiological parameters of the patient monitored via an implantable medical device (IMD). A patient's stroke risk may be reflected by a stroke risk score generated by a stroke risk monitoring system including the IMD. The risk monitoring system may generate a patient stroke risk score based on patient stroke risk factors, such as, e.g., atrial fibrillation, hypertension, congestive heart failure (CHF), diabetes, and the like, identified by the monitoring system as being present in a patient. The risk monitoring system may detect the presence of one or more stroke risk factors used to compute a stroke risk score of the patient based on one or more physiological parameters of the patient monitored via the implantable medical device. The risk monitoring system may monitor for the presence of a plurality of stroke risk factors used to determine the stroke risk score a patient to track a patient's risk of stroke. In some examples, the stroke risk monitoring system may alert the patient or a clinician based on the stroke risk score determined by the monitoring system, e.g., when the patient stroke risk score indicates an elevated stroke risk for the patient.

The stroke risk of a patient may be estimated based on the presence or absence of one or more stroke risk factors. In general, a stroke risk factor is a factor which is known to correlate to an increased likelihood of stroke in a patient. Examples of stroke risk factors may include, but are not limited to, hypertension, congestive heart failure, diabetes mellitus, prior stroke or transient ischemic attack, atrial fibrillation, high blood cholesterol, obesity, sickle cell disease, and the like. When one or more stroke risk factors are present in a patient, the patient may generally be considered to have a greater risk of a stroke compared to a patient with no stroke risk factors present. In some cases, certain stroke risk factors may correlate to a greater risk of stroke relative to other stroke risk factors.

Depending on the overall stroke risk of a patient indicated by the presence (or absence) of one or more stroke risk factors for a patient, treatment may be provided to a patient having a high stroke risk to combat the risk of stroke. For example, after determining that a patient has a relatively high risk of stroke using one or more established stroke risk assessment tools, a clinician may prescribe an anticoagulant, such as warfarin, to a patient to effectively reduce the risk of ischemic stroke. In other examples, the stroke risk factor itself may be treated directly, e.g., an obese patient may modify his/her diet to lose weight.

However, while a number of effective treatments are available for patients having a high risk of stroke, such treatments are not always timely prescribed due in part to the lack of timely identification of one or more stroke risk factors in a patient. In some situations, a clinician may only evaluate a patient for the presence of one or more stroke risk factors, if at all, during scheduled patient check-ups. Depending on the frequency of the scheduled check-ups, there may be extended periods of time between check-ups during which a patient may exhibit one or more new stroke risk factors without the clinician's knowledge. In such a scenario, the stroke risk of a patient may be at an undesirable level for a relatively long amount of time without being identified as such by a clinician. Thus, the stroke risk of patient may go untreated until the undesirable level of stroke risk is identified by a clinician, which may be an extended period of time. Furthermore, for a patient being treated for elevated stroke risk, e.g., via anticoagulants, such treatment may continue even after the patient stroke risk decreases, thereby unnecessarily treating a patient because the reduction in stroke risk was not identified by a clinician.

As will be described in further detail below, in some examples, the stroke risk of a patient may be tracked via a stroke risk monitoring system. To track the stroke risk of the patient, the stroke risk monitoring system may include an IMD configured to monitor one or more physiological parameters of the patient via a sensing module. Based at least in part on the physiological parameter(s) monitored by the IMD, the system may determine whether one or more of a plurality of stroke risk factors are present in the patient. For example, the system may detect the presence of hypertension based on blood pressure information sensed by a pressure sensor of the IMD and/or the presence of atrial fibrillation based on electrical activity sensed by the IMD via one or more electrodes implanted in the heart of the patient.

Based on the one or more stroke risk factors identified as being present in the patient, the system may compute a stroke risk score for the patient. The stroke risk score generated by the stroke risk monitoring system may be reflective of a patient's overall risk of stroke, as indicated by the stroke risk factor(s) present in the patient for the time frame the stroke risk score was computed. By tracking the stroke risk score of a patient over a period of time, e.g., by generating a stroke risk score on a continuous or periodic basis, the stroke risk monitoring system can identify changes to the stroke risk of a patient; especially changes to a patient's stroke risk score which reflect an increased risk of the patient to stroke.

In general, the stroke risk score generated by the stroke risk monitoring system based on the presence of stroke risk factor(s) identified by monitoring one or more physiological parameters of a patient via an IMD reflects the patient's risk of stroke. By tracking the stroke risk score of a patient, the monitoring system may be able to track the relative stroke risk of a patient over time. In some examples, the monitoring system may compute the stroke risk score in terms of a numerical value. For example, individual stroke risk factors may be assigned a numerical factor value that correlates with the degree of stroke risk associated with the stroke risk factor. In some examples, all stroke risk factors monitored by the stroke risk monitoring system may be assigned substantially the same numerical value. In other examples, the values may be assigned to each stroke risk factor may be weighted based on the degree of risk associated with the presence of each particular stroke risk factor. In each case, the stroke risk monitoring system may compute the stroke risk score as the aggregate of all the values assigned to the stroke risk factors identified as being present at that time or according to another suitable methodology. Again, one or more of the patient stroke risk factors may be identified as being present based on one or more physiological parameters monitored via an IMD.

In other examples, more complicated algorithms may be employed to compute a stroke risk score based on the presence of stroke risk factor(s) in a patient. For example, the presence of certain combinations of stroke risk factors may be assigned greater weight than compared to the total of the stroke risk parameters calculated individually. In some cases, the monitoring system may compute a stroke risk score in terms of abstract risk stratification levels. Based on the presence or absence of one or more stroke risk factors, the risk monitoring system may assign the patient into one or more stroke risk strata (e.g., high risk, medium risk, low risk).

Regardless of the particular methodology used to compute the stroke risk score, the stroke risk score generated by the stroke risk monitoring system may accurately reflect the actual stroke risk of a patient indicated by the presence or absence of one or more stroke risk factors. Accordingly, by tracking the stroke risk score of the patient, the stroke monitoring system may be able to identify the relative stroke risk of the patient, as well as changes to the stroke risk of a patient that may occur over a period of time. If a patient develops a stroke risk factor, e.g., hypertension, not previously present in the patient, then the change in patient situation may be reflected by a change in the stoke risk score computed by the monitoring system, e.g., the stroke risk score may increase upon identification of the new stroke risk factor, at least to the extent that the presence of the one or more stroke risk factors actually increases the patient's risk of stroke. Similarly, the stroke risk score computed by the stroke risk monitoring system may reflect the absence of a stroke risk factor that was previously present for a patient with a decrease in the patient's stroke risk score.

In some examples, the stroke risk monitoring system may be configured to alert a user, e.g., a clinician and/or patient, based on the stroke risk score generated by the system. For example, the stroke risk monitoring system may alert a user when the stroke risk score computed by the stroke risk monitoring system reaches some preset threshold value. In other examples, the stroke risk monitoring system may alert a user upon a change to the stroke risk score computed by the monitoring system (e.g., any increase or decrease in the stroke risk score). In this manner, using an IMD to monitor one or more physiological parameters of a patient, the stroke risk monitoring system may track the stroke risk of a patient, and alert a user to increased, decreased and/or untenable stroke risk developed by a patient. By monitoring the stroke risk of the patient using such a monitoring system, the development of a relatively high stroke risk may be identified within a relatively short period of time and, thus, may be addressed in a timely manner, e.g., via the prescription of anticoagulants by a clinician. Similarly, a reduction in the stroke risk of a patient may be identified within a relatively short period of time and, thus, may be addressed in a timely manner, e.g., by terminating or adjusting the prescription of anticoagulants to the patient to be consistent with the reduced stroke risk.

According to some examples of the disclosure, the presence of one or more patient stroke risk factors may be detected based on one or more physiological parameters monitored by an IMD. For example, the IMD may be configured to sense blood pressure, electrical activity, e.g., cardiac electrical activity, blood sugar levels, and/or other physiological parameters that may be suitable for identifying the presence of one or more stroke risk factors. The stroke monitoring system may analyze the physiological parameters monitored by the IMD to detect whether or not a stroke risk factor is present in patient. Example stroke risk factors include, but are not limited to, hypertension, congestive heart failure, diabetes mellitus, prior stroke or transient ischemic attack, atrial fibrillation, high blood cholesterol, obesity, sickle cell disease, and the like.

In some examples, the stroke risk monitoring system may detect the presence of one or more stroke risk factors based on information other than that of the monitored patient physiological parameters. For example, the stroke risk monitoring system may detect the presence of one or more stroke risk factors (e.g., patient sex, weight, age) based on information indicated by a user such as the patient or clinician. The monitoring system may use the user identified stroke risk factors to compute a stroke risk score, in addition to one or more stroke risk factors identified using physiological parameters monitored by the IMD.

A stroke risk monitoring system may include one or more external devices, such as, e.g., an external programming device, that is in communication with the IMD. In some examples, the IMD may determine whether each of a plurality of stroke risk factors are present and then compute the stroke risk score in addition to monitoring the one or more physiological parameters. In other examples, the IMD may monitor the one or more physiological parameters, and transmit data representative of the parameters to an external computing device, such as an IMD programmer. The external computing device may then determine whether each of a plurality of stroke risk factors are present and then compute the stroke risk score of the patient that is based at least in part on the stroke risk factors present for the patient. In some examples, the IMD and external device may determine the presence of stroke risk factors and compute the stroke risk score in combination with one another.

In some examples, the IMD that monitors the one or more physiological parameters used to identify stroke risk factor(s) may be configured to perform additional functions within the patient. For example, the IMD may be configured to deliver stimulation therapy to the patient in addition to monitoring physiological parameters of the patient for the identification of stroke risk factors. In one example, the IMD may deliver cardiac therapy (e.g., at least one of pacing, cardioversion, and defibrillation stimulation therapy) to the heart of the patient. In such an example, the IMD may monitor one or more of the physiological parameters to provide cardiac therapy to the patient in addition to identifying the presence of patient stroke risk factors used to compute a patient stroke risk score.

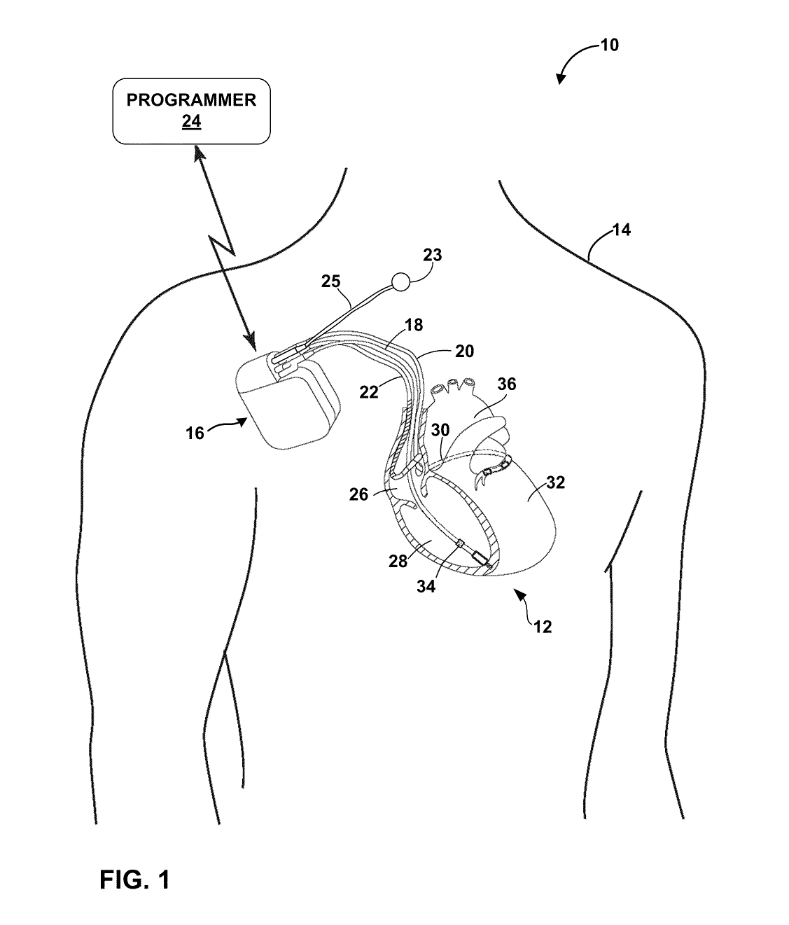

FIG. 1 is a conceptual diagram illustrating an example stroke risk monitoring system 10 that may be used to monitor the stroke risk of patient 14. System 10 includes IMD 16, which is coupled to leads 18, 20, and 22, physiological parameter sensor 23, and programmer 24. In some examples, IMD 16 may be, for example, an implantable pacemaker, cardioverter, and/or defibrillator that provides electrical signals to heart 12 via electrodes coupled to one or more of leads 18, 20, and 22. Alternatively or additionally, IMD 16 may be configured to deliver non-cardiac therapy to patient 14, such as, e.g., neurostimulation therapy. In other examples, IMD 16 may be an implantable monitoring device for monitoring one or more physiological parameters of patient 14 that does not also provide therapy (e.g., stimulation therapy) to patient 14.

As will described in further detail below, stroke risk monitoring system 10 may be used to monitor the stroke risk of patient 14 by computing a stroke risk score. To compute the stroke risk score of patient 14, system 10 may determine the presence of various stroke risk factors based at least in part on one or more physiological parameters of patient 14 monitored by IMD 16, such as, e.g., blood pressure or blood flow of patient 14, cardiac signals of a heart 12 of patient 14, intrathoracic impedance, and/or blood sugar level of patient 14 IMD 16 monitors the physiological parameters via the electrodes coupled to leads 18, 20, 22, or one or more sensors, such as physiological parameter sensor 23 and/or pressure sensor 34. The stroke risk factors identified by system 10 may be factors that correlate with an increased risk of stroke for patient 14. As system 10 may be configured to detect the presence of multiple stroke risk factors in patient 14, the stroke risk score computed by system 10 may be based on the stroke risk factor(s) identified in patient 14.

Leads 18, 20, 22 extend into the heart 12 of patient 16 to, for example, sense electrical activity of heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of the surface of the left ventricle 32 of heart 12. Right atrial (RA) lead 22 extends through one or more veins and the vena cava, and into the right atrium 26 of heart 12.

IMD 16 may sense electrical signals attendant to the depolarization and repolarization of heart 12 via electrodes (not shown in FIG. 1) coupled to at least one of the leads 18, 20, 22. The configurations of electrodes used by IMD 16 for sensing may be unipolar (e.g., using a lead electrode and a can electrode) or bipolar (e.g., using two lead electrodes). IMD 16 may collect, for example, cardiac signals in the form of an electrogram (EGM). IMD 16 may also provide defibrillation therapy and/or cardioversion therapy via electrodes located on at least one of the leads 18, 20, 22. IMD 16 may detect tachyarrhythmia of heart 12, such as fibrillation of ventricles 28 and 32, and deliver defibrillation therapy to heart 12 in the form of electrical pulses. In some examples, IMD 16 may be programmed to deliver a progression of therapies, e.g., pulses with increasing energy levels, until a fibrillation of heart 12 is stopped. IMD 16 detects tachycardia and fibrillation employing one or more tachycardia and fibrillation detection techniques known in the art.

In accordance with some examples of the disclosure, IMD 16 may be configured to monitor one or more physiological parameters of patient to identify the presence of one or more stroke risk factors. System 10 may generate a stroke risk score that is based at least part on one or more stroke risk factors identified based on the physiological parameters monitored by IMD 16. One or more physiological sensors, such as, e.g., pressure sensor 34 and physiological parameter sensor 23, may be coupled to a sensing module of IMD 16 to monitor physiological parameters which may be indicative of one or more stroke risk factors.

In some examples, IMD 16 may sense electrical signals of heart 12 via electrodes coupled to one or more of leads 18, 20, 22 to detect the presence of one or more patient stroke risk factors. For examples, IMD 16 may monitor the electrical activity heart 12 via one or more electrodes on leads 18, 20, and/or 22 to detect the presence of atrial fibrillation in patient 14. IMD 16 may detect the presence of atrial fibrillation based on the monitored electrical signals using any suitable methodology. System 10 may generate a stroke risk score that is based at least in part on the presence or absence of atrial fibrillation in patient 14. In general, the stroke risk of patient 14 increases when atrial fibrillation is detected in heart 12 of patient 14. Thus, upon identification of the presence of atrial fibrillation in patient 14, the stroke risk score computed by IMD 16, programmer 24, or other computing device may reflect an increase in patient stroke risk.

One or more of leads 18, 20, 22 may also carry a pressure sensor 34. Pressure sensor 34 may be used by IMD 16 to monitor pressure within heart 12 of patient 14. In the example illustrated in FIG. 1, pressure sensor 34 is attached adjacent a distal end of lead 18 and positioned in right ventricle 28. Pressure sensor 34 may respond to an absolute pressure inside right ventricle 28, and may be, for example, a capacitive sensor, piezoelectric sensor, mechanical sensor, fiber optic sensor, or the like. In other examples, pressure sensor 34 may be positioned within other regions of heart 12 and may monitor pressure within one or more of the other regions of heart 12, or may be positioned elsewhere within or proximate to the cardiovascular system of patient 14 to monitor cardiovascular pressure associated with mechanical contraction of the heart 12. For example, pressure sensor 34 may be positioned within right atrium 26, left atrium 30, left ventricle 32, or a vein or artery. An example of a suitable pressure sensor may include that associated with the Chronicle Implantable Hemodynamic Monitor manufactured by Medtronic, Inc. of Minneapolis, Minn.

Placement of pressure sensor 34 in right ventricle 28 may enable measurement of a variety of hemodynamic parameters by IMD 16. For example, pressure sensor 34 may be used to detect right ventricular (RV) systolic and diastolic pressures (RVSP and RVDP), estimated pulmonary artery diastolic pressure (EPAD), and pressure changes with respect to time (dP/dt). Some parameters may be derived from other parameters, rather than being directly detected by pressure sensor 34. For example, the EPAD parameter may be derived from RV pressure at the moment of pulmonary valve opening. System 10 may detect the presence of one or more stroke risk factors, such as, e.g., hypertension or CHF, based on the hemodynamic parameters monitored via pressure sensor 34.

Pressure sensor 34 in the example of FIG. 1 may be used to detect pressure data relating to right ventricular (RV) pressure. In other examples, however, it is contemplated that any type of sensor could be used, such as a self-contained implantable pressure sensor or a flow sensor in the venous or arterial system. Further, the blood pressure can be detected in other locations of patient 14, including other chambers of heart 12. For example, pressure sensor 34 may be positioned to detect, for example, a left ventricular systolic pressure (LVSP), a left ventricular diastolic pressure (LVDP), a left ventricular pulse pressure (LVPP), a left atrial pressure (LAP), or a right atrial pressure (RAP), in various example implementations.

In some examples, IMD 16 may monitor pressure at one or more sites within patient 14 via pressure sensor 34 to identify the presence of one more stroke risk factors. For example, pressure information generated by pressure sensor 34 may be used to identify the presence of hypertension within patient 14, which may be considered a stroke risk factor. In some examples, the mean pressure in one or more arteries detected via pressure sensor 34 may be used to identify the presence of hypertension. The presence or absence of hypertension in patient 14 may influence the stroke risk score of patient 14 generated by system 10. In particular, the stroke risk score computed by system 10 may reflect an increased risk of stroke for patient 14 when hypertension has been identified in patient 14. Similarly, IMD 16 may monitor pressure at one or more sites within patient 14 via pressure sensor 34 (e.g., EPAD) to identify the presence of congestive heart failure, which may be considered a stroke risk factor. The presence or absence of congestive heart failure in patient 14 may influence the stroke risk score of patient 14 generated by system 10. In particular, the stroke risk score computed by system 10 may reflect an increased risk of stroke for patient 14 when congestive heart failure has been identified in patient 14.

Additionally or alternatively, IMD 16 may sense one or more physiological parameter of patient 14 via physiological parameter sensor 23, which may be coupled to a sensing module within IMD 16 via lead 25. In FIG. 1, unlike that of pressure sensor 34, physiological parameter sensor 23 is shown located outside heart 12 of patient 14. As such, physiological parameters sensor 23 may be configured sense one or more physiological parameters of patient 14 outside of heart 12. In other examples, sensor 23 may also be located within or proximate heart 12 of patient 14. In some examples, physiological parameter sensor 23 may be located on one of leads 18, 20, 22 instead of the separate lead 25 in the illustrated example. In some examples, physiological parameter sensor 23 may include multiple sensors each capable of sensing the same or different physiological parameters of a patient 14. In each case, sensor information generated by sensing 23 may allow IMD 16 to monitor one or more physiological parameters of patient 14 in a manner that allows system 10 to identify the presence of one or more stroke risk factors in patient 14. For example, IMD 14 may monitor the blood sugar level of patient 14 via sensor 34 to detect the presence of diabetes mellitus in patient 14. Additionally or alternatively, sensor 34 may be used to monitor the physical activity of patient 14, e.g., via one or more accelerometers for sensing patient movement. In some examples, sensor 34 may be used to monitor blood cholesterol level and/or blood hematocrit within patient 14.

In some examples, programmer 24 may be a handheld computing device or a computer workstation. Programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some examples, a display of programmer 24 may include a touch screen display, and a user may interact with programmer 24 via the display.

A user, such as patient 14, a physician, technician, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., to select values for operational parameters of the IMD 16.

For example, a user such as a clinician may use programmer 24 to retrieve information from IMD 16 regarding the rhythm of heart 12, trends therein over time, or tachyarrhythmic episodes. As another example, the user may use programmer 24 to retrieve information from IMD 16 regarding other sensed physiological parameters of patient 14, such as intracardiac or intravascular pressure, activity, posture, respiration, thoracic impedance, blood sugar levels, and the like. As a further example, the user may use programmer 24 to retrieve information from IMD 16 regarding the performance or integrity of IMD 16 or other components of system 10, such as leads 18, 20, and 22, or a power source of IMD 16.

In some examples, programmer 24 may also receive alerts from IMD 16, such as an alert generated in response to the determination by IMD 16 of a stroke risk score which correlates to an increased, decreased, and/or undesirable stroke risk to patient 14. Programmer 24 may also compute patient stroke risk scores and/or generate alerts based on the stroke risk score based on information received from IMD 16, e.g., physiological parameter information and/or stroke risk factor information. In some examples, a user may use programmer 24 to retrieve information from IMD 16 regarding physiological parameter(s) monitored by IMD 16, to retrieve information indicating the presence of stroke risk factor detected by IMD 16, and/or to retrieve information regarding one or more stroke risk scores of patient 14 generated by IMD 16.

The user may use programmer 24 to program a therapy progression, select electrodes used to deliver cardioversion or defibrillation pulses, select waveforms for the cardioversion or defibrillation pulses, or select or configure a tachyarrhythmia detection algorithm for IMD 16. The user may also use programmer 24 to program aspects of other therapies provided by IMD 14, such as pacing therapies.

In some examples, a user may use programmer 24 to indicate the presence of one or more stroke risk factors identified without the physiological parameter information monitored by IMD 16, or patient information that may be used by system 10 to identify one or more stroke risk factors without the use of the physiological parameter information monitored by IMD 16. For example, a user may indicate the sex of patient 14, age of patient 14 to IMD 16 or other device of system 10 using programmer 24 and/or the occurrence of a prior stroke or transient ischemic attack (TIA) to system 10 (e.g., if such occurrences were not identified by IMD 16). Using patient age information, system 10 may track the age of patient to identify when patient 14 is beyond a threshold age which defines a stroke risk factor. In some example, if patient 14 is older than 75 years, for example, patient age may be considered a stroke risk factor. In some examples, patient age may be stratified to define patient stroke risk factors. For example, a first stroke risk factor may be defined as the age of a patient being over 75 years old, and a second stroke risk factor may be defined as the age of a patient being between 65 and 75 years old. In each case, the first and second strata are both stroke risk factors but each stroke risk factor may be treated differently in terms of computing a stroke risk score, e.g., an age above 75 years old may correlate to a higher risk of stroke than an age between 65 and 75 years old. In some examples, system 20 may treat age as a continuous variable, e.g., such that age 80 is a higher stroke risk than age 75 but a lower stroke risk than age 85. The information provided by a user to system 10 relating stroke risk factors of a patient may be used by system 10 to identify the presence of one or more stroke risk factors. The presence of such stroke risk factor may be used by system 10 to generate a stroke risk score for patient 14, in addition to the presence of one or more stroke risk factors detected by system 10 by monitoring one or more physiological parameters of patient 14 via IMD 16.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the body of patient 14 near the IMD 16 implant site in order to improve the quality or security of communication between IMD 16 and programmer 24.

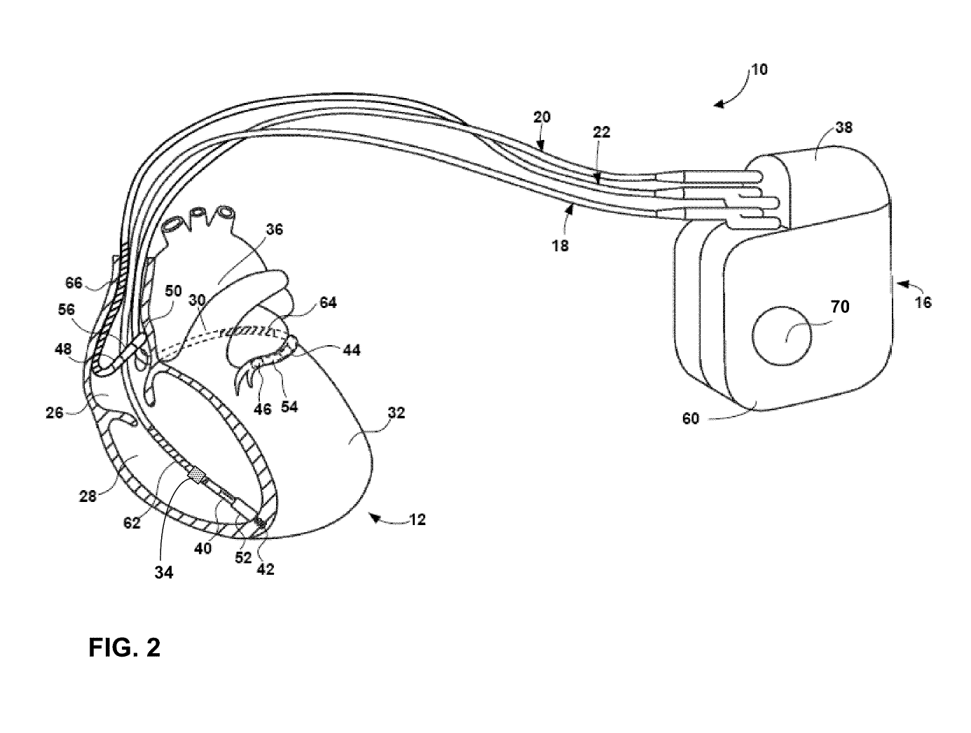

FIG. 2 is a conceptual diagram illustrating IMD 16 and leads 18, 20, and 22 of therapy system 10 in greater detail. Physiological parameter sensor 23 and lead 25 are not shown in FIG. 2, for ease of illustration. Leads 18, 20, 22 may be electrically coupled to a signal generator, a sensing module, or other modules of IMD 16 via connector block 34. In some examples, proximal ends of leads 18, 20, 22 may include electrical contacts that electrically couple to respective electrical contacts within connector block 34 of IMD 16. In addition, in some examples, leads 18, 20, 22 may be mechanically coupled to connector block 34 with the aid of set screws, connection pins, snap connectors, or another suitable mechanical coupling mechanism.

Each of the leads 18, 20, 22 includes an elongated insulative lead body, which may carry a number of concentric coiled conductors separated from one another by tubular insulative sheaths. In the illustrated example, pressure sensor 34 and bipolar electrodes 40 and 42 are located adjacent to a distal end of lead 18 in right ventricle 28. In addition, bipolar electrodes 44 and 46 are located adjacent to a distal end of lead 20 in coronary sinus 30 and bipolar electrodes 48 and 50 are located adjacent to a distal end of lead 22 in right atrium 26. There are no electrodes located in left atrium 36, but other embodiments may include electrodes in left atrium 36. In FIG. 2, pressure sensor 34 is again disposed in right ventricle 28 for purposes of illustration. Pressure sensor 34 may respond to an absolute pressure inside right ventricle 28, and may be, for example, a capacitive sensor, piezoelectric sensor, mechanical sensor, fiber optic sensor, or the like. In other examples, pressure sensor 34 may be positioned within other regions of heart 12 and may monitor pressure within one or more of the other regions of heart 12, or may be positioned elsewhere within or proximate to the cardiovascular system of patient 14 to monitor cardiovascular pressure associated with mechanical contraction of the heart. In some examples, pressure information sensed by IMD 16 via pressure sensor 34 may be used by system 10 to detect the presence of one or more patient stoke risk factors, e.g., hypertension or CHF.

Electrodes 40, 44 and 48 may take the form of ring electrodes, and electrodes 42, 46 and 50 may take the form of extendable helix tip electrodes mounted retractably within insulative electrode heads 52, 54 and 56, respectively. In other embodiments, one or more of electrodes 42, 46 and 50 may take the form of small circular electrodes at the tip of a tined lead or other fixation element. Leads 18, 20, 22 also include elongated electrodes 62, 64, 66, respectively, which may take the form of a coil. Each of the electrodes 40, 42, 44, 46, 48, 50, 62, 64 and 66 may be electrically coupled to a respective one of the coiled conductors within the lead body of its associated lead 18, 20, 22, and thereby coupled to respective ones of the electrical contacts on the proximal end of leads 18, 20 and 22.

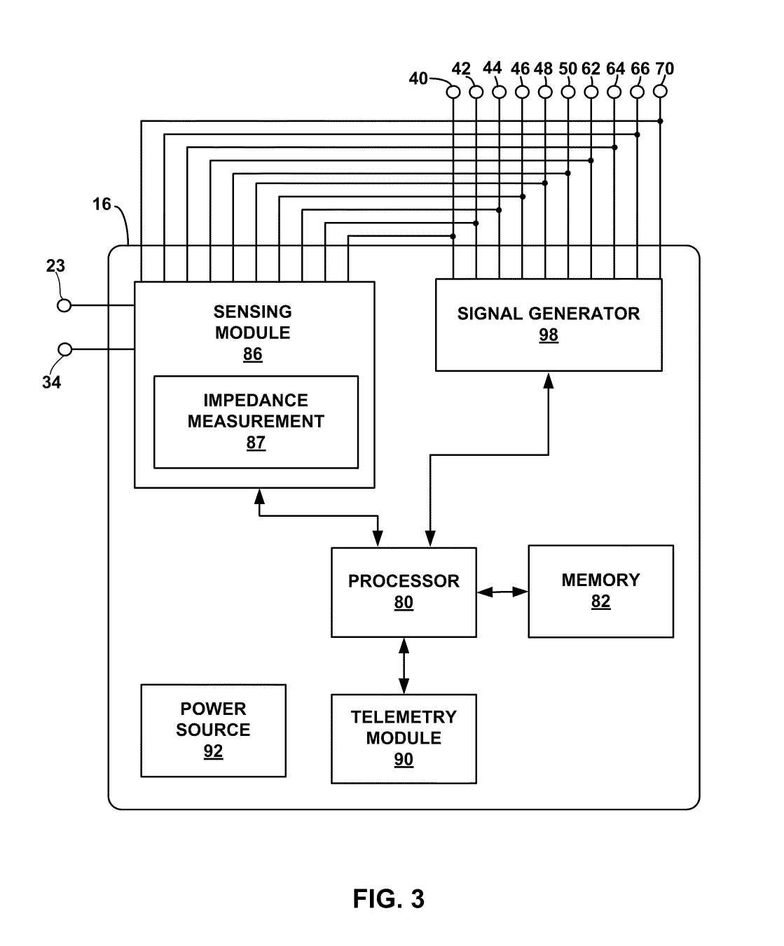

In some examples, as illustrated in FIG. 2, IMD 16 includes one or more housing electrodes, such as housing electrode 58, which may be formed integrally with an outer surface of hermetically-sealed housing 60 of IMD 16 or otherwise coupled to housing 60. In some examples, housing electrode 58 is defined by an uninsulated portion of an outward facing portion of housing 60 of IMD 16. Other division between insulated and uninsulated portions of housing 60 may be employed to define two or more housing electrodes. In some examples, housing electrode 58 comprises substantially all of housing 60. As described in further detail with reference to FIG. 3, housing 60 may enclose a stimulation generator that generates therapeutic stimulation, such as cardiac pacing pulses and defibrillation shocks, as well as a sensing module for monitoring the rhythm of heart 12 and/or other physiological parameters of patient 14.

IMD 16 may sense electrical signals attendant to the depolarization and repolarization of heart 12 via electrodes 40, 42, 44, 46, 48, 50, 62, 64 and 66. The electrical signals are conducted to IMD 16 from the electrodes via the respective leads 18, 20, 22. IMD 16 may sense such electrical signals via any bipolar combination of electrodes 40, 42, 44, 46, 48, 50, 62, 64 and 66. Furthermore, any of the electrodes 40, 42, 44, 46, 48, 50, 62, 64 and 66 may be used for unipolar sensing in combination with housing electrode 58. In some examples, any of electrodes 40, 42, 44, 46, 48, 50, 62, 64, 66, and 58 may be used for monitoring electrical activity of heart 12 to detect the presence of one or more patient stroke risk factors, such as, e.g., atrial fibrillation, and the like.

In some examples, IMD 16 delivers pacing pulses via bipolar combinations of electrodes 40, 42, 44, 46, 48 and 50 to produce depolarization of cardiac tissue of heart 12. In some examples, IMD 16 delivers pacing pulses via any of electrodes 40, 42, 44, 46, 48 and 50 in combination with housing electrode 58 in a unipolar configuration. Furthermore, IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes.

Pressure sensor 34 may be coupled to one or more elongated, coiled conductors within lead 42. In FIG. 2, pressure sensor 34 is located more distally on lead 18 than elongated electrode 74. In other examples, pressure sensor 34 may be positioned more proximally than elongated electrode 74, rather than distal to electrode 74. Further, pressure sensor 34 may be coupled to another one of the leads 44, 46 in other examples, or to a lead other than leads 42, 44, 46 carrying stimulation and sense electrodes. In addition, in some examples, pressure sensor 34 may be self-contained device that is implanted within heart 12, such as within the septum separating right ventricle 28 from left ventricle 32, or the septum separating right atrium 26 from left atrium 33. In such an example, pressure sensor 34 may wirelessly communicate with IMD 16. Similarly, physiological parameter sensor 23 (FIG. 1) may wirelessly communicate with IMD 16.

System 10 may include any suitable number of leads coupled to IMD 16, and each of the leads may extend to any location within or proximate to heart 12. For example, other examples of therapy systems may include three transvenous leads located as illustrated in FIGS. 1 and 2, and an additional lead located within or proximate to left atrium 36. As another example, other examples of therapy systems may include a single lead that extends from IMD 16 into right atrium 26 or right ventricle 28, or two leads that extend into a respective one of the right ventricle 26 and right atrium 26. An example of this type of therapy system is shown in FIG. 3. Any electrodes located on these additional leads may be used in sensing configurations that may be pre-qualified as described herein.

The configurations of monitoring system 10 illustrated in FIGS. 1 and 2 is merely one example. In other examples, a monitoring system or therapy system may include epicardial leads and/or patch electrodes instead of or in addition to the transvenous leads 18, 20, 22, illustrated in FIGS. 1 and 2. In other examples of therapy systems that provide electrical stimulation therapy to heart 12, a therapy system may include any suitable number of leads coupled to IMD 16, and each of the leads may extend to any location within or proximate to heart 12. For example, other examples of therapy systems may include three transvenous leads located as illustrated in FIGS. 1 and 2, and an additional lead located within or proximate to left atrium 33. As another example, other examples of therapy systems may include a single lead that extends from IMD 16 into right atrium 26 or right ventricle 28, or two leads that extend into a respective one of the right ventricle 26 and right atrium 28. As indicated above, in some examples, IMD 16 may be configured as a monitoring device not configured to delivery stimulation therapy to heart 12 of patient 14 or may be a monitoring device that is also configured to deliver stimulation therapy other than cardiac stimulation therapy, e.g., neurostimulation therapy. In some cases, IMD 16 may be a subcutaneous monitoring device that may or may not have one or more leads within heart 12. In some examples, IMD 16 need not include leads, and instead may include a plurality of electrodes located on or formed integrally with a housing of the IMD. One example of such a device is the Reveal® monitoring device commercially available from Medtronic, Inc., of Minneapolis, Minn.

FIG. 3 is a functional block diagram of one example configuration of IMD 16, which includes a processor 80, memory 82, sensing module 86, telemetry module 90, power source 92, and signal generator 98. Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, magneto-resistive random access memory (MRAM), or any other digital media. In some examples, memory 82 may include instructions for detecting the presence of one or more stroke risk based on one or more physiological parameters monitored via sensing module 86 and/or instructions for computing a stroke risk score of patient 14 as described herein.

Processor 80 may include any one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof.

In an examples in which IMD 16 is configured to deliver therapy, processor 80 controls signal generator 98 to deliver stimulation therapy to heart 12 according to a selected one or more of therapy programs, which may be stored in memory 82. Specifically, processor 44 may control signal generator 98 to deliver electrical pulses with the amplitudes, pulse widths, frequency, or electrode polarities specified by the selected one or more therapy programs. Signal generator 98 is electrically coupled to electrodes 40, 42, 44, 46, 48, 50, 58, 62, 64, and 66, e.g., via conductors of the respective lead 18, 20, 22, or, in the case of housing electrode 70, via an electrical conductor disposed within housing 60 of IMD 16.

Signal generator 98 may be configured to generate and deliver electrical stimulation therapy to heart 12. For example, signal generator 98 may deliver defibrillation shocks to heart 12 via at least two electrodes 58, 62, 64, 66. Signal generator 98 may deliver pacing pulses via ring electrodes 40, 44, 48 coupled to leads 18, 20, and 22, respectively, and/or helical electrodes 42, 46, and 50 of leads 18, 20, and 22, respectively. In some examples, signal generator 98 delivers pacing, cardioversion, or defibrillation stimulation in the form of electrical pulses. In other examples, therapy module 98 may deliver one or more of these types of stimulation in the form of other signals, such as sine waves, square waves, or other substantially continuous time signals.

Signal generator 98 may include a switch module and processor 80 may use the switch module to select, e.g., via a data/address bus, which of the available electrodes are used to deliver defibrillation pulses or pacing pulses. The switch module may include a switch array, switch matrix, multiplexer, or any other type of switching device suitable to selectively couple stimulation energy to selected electrodes.

Sensing module 86 monitors signals from at least one of electrodes 40, 42, 44, 46, 48, 50, 58, 62, 64 or 66 in order to monitor electrical activity of heart 12. Sensing module 86 may also include a switch module to select which of the available electrodes are used to sense the heart activity. In some examples, processor 80 may select the electrodes that function as sense electrodes, or the sensing configuration, via the switch module within sensing module 86, e.g., by providing signals via a data/address bus. Sensing module 86 includes multiple detection channels, each of which may comprise an amplifier. In another example, sensing module 86 may only include one detection channel to sensing cardiac signals that may then be digitized and processed by processor 80 to analyze the signal detected by multiple sensing configurations. In response to the signals from processor 80, the switch module of within sensing module 86 may couple selected electrodes to one of the detection channels.

In some examples, sensing module 86 monitors the electrical activity of heart 12 to detect the presence of one or more stroke risk factors in patient 14. For example, processor 80 may detect the presence of atrial fibrillation (or other atrial tachyarrhythmia) in heart 12 based on the electrical activity of heart 12 monitored via sensing module 86 using one or more suitable methodologies. Similarly, sensing module 86 may monitor signals from at least one of pressure sensor 34 and physiological parameter sensor 23 to detect the presence of one or more stroke risk factors in patient 14. For examples, processor 80 may detect the presence of hypertension in patient 14 based on the signals generated by pressure sensor 34 using one or more suitable detection methodologies. As another example, processor 80 may detect the presence of diabetes in patient 14 based on the signals generated by physiological sensor 23 using one or more suitable detection methodologies. The stroke risk score generated by system 10 may reflect the presence or absence of the one or more stroke risk factors detected via sensing module 86 to monitor the stroke risk of patient 14. Stroke risk factors that may be detected based on the electrical activity of heart 12 sensed by IMD 14 via sensing module 86 may include, but are not limited to, atrial fibrillation, congestive coronary artery disease, myocardial infarction, atrial flutter, atrial tachycardia, ventricular dysfunction (e.g., based on ventricular heart rate), and the like.

As illustrated in FIG. 3, sensing module 86 may include an impedance measurement module 87. Processor 80 may control impedance measurement module 87 to periodically measure an electrical parameter to determine an impedance, such as a intrathoracic impedance. By monitoring intrathoracic impedance via sensing module 86, system 10 may detect the presence of congestive heart failure, which may be considered a stroke risk factor. Such techniques may be used to detect congestive heart failure alone or in conjunction with those techniques utilizing other physiological parameters to detect the presence of congestive heart failure in patient 14. In some examples, sensing module 86 may monitor respiration generally, or nighttime respiration, which may be determined via intrathoracic impedance or noise on a cardiac EGM signal, to detect congestive heart failure. In other examples, system 10 may identify the presence congestive heart failure by measuring intracardiac flow via impedance measurements by impedance measurement module 87.

For intrathoracic impedance measurement, processor 80 may control stimulation generator 84 to deliver an electrical signal between selected electrodes and impedance measurement module 87 to measure a current or voltage amplitude of the signal. Processor 80 may select any combination of electrodes 40, 42, 44, 46, 48, 50, 62, 64, 66, and 70 e.g., by using switch modules in signal generator 98 and sensing module 86. Impedance measurement module 87 may include sample and hold circuitry or other suitable circuitry for measuring resulting current and/or voltage amplitudes. Processor 80 determines an impedance value from the amplitude value(s) received from impedance measurement module 87.

In some examples, processor 80 may perform an impedance measurement by causing signal generator 84 to deliver a voltage pulse between two electrodes and examining resulting current amplitude value measured by impedance measurement module 87. In these examples, signal generator 84 delivers signals that do not necessarily deliver stimulation therapy to heart 12, due to, for example, the amplitudes of such signals and/or the timing of delivery of such signals. For example, these signals may comprise sub-threshold amplitude signals that may not stimulate heart 12. In some cases, these signals may be delivered during a refractory period, in which case they also may not stimulate heart 12.

In other examples, processor 80 may perform an impedance measurement by causing signal generator 84 to deliver a current pulse across two selected electrodes. Impedance measurement module 87 holds a measured voltage amplitude value. Processor 80 determines an impedance value based upon the amplitude of the current pulse and the amplitude of the resulting voltage that is measured by impedance measurement module 87. IMD 16 may use defined or predetermined pulse amplitudes, widths, frequencies, or electrode polarities for the pulses delivered for these various impedance measurements. In some examples, the amplitudes and/or widths of the pulses may be sub-threshold, e.g., below a threshold necessary to capture or otherwise activate tissue, such as cardiac tissue.

In certain cases, IMD 16 may measure intrathoracic impedance values that include both a resistive and a reactive (i.e., phase) component. In such cases, IMD 16 may measure impedance during delivery of a sinusoidal or other time varying signal by signal generator 84, for example. Thus, as used herein, the term “impedance” is used in a broad sense to indicate any collected, measured, and/or calculated value that may include one or both of resistive and reactive components.

In the example illustrated in FIG. 3, processor 80 and/or impedance analysis unit 92 are capable of performing the various techniques described in FIG. 1. To avoid confusion, impedance analysis unit 92 is described as performing the various impedance processing techniques proscribed to IMD 16, but it should be understood that these techniques may also be performed by processor 80. Although processor 80 and impedance analysis unit 92 are illustrated as separate modules in FIG. 3, processor 80 and impedance analysis unit 92 may be incorporated in a single processing unit.

Processor 80 may maintain programmable counters. For example, if IMD 16 is configured to generate and deliver pacing pulses to heart 12, processor 80 may maintain programmable counters which control the basic time intervals associated with various modes of pacing, including pacing for cardiac resynchronization therapy (CRT) and anti-tachycardia pacing (ATP). Such intervals may include atrial and ventricular pacing escape intervals, refractory periods during which sensed P-waves and R-waves are ineffective to restart timing of the escape intervals, and the pulse widths of the pacing pulses. As another example, processor 80 may define a blanking period, and provide signals sensing module 86 to blank one or more channels, e.g., amplifiers, for a period during and after delivery of electrical stimulation to heart 12. The durations of these intervals may be determined by processor 80 in response to stored data in memory 82. Processor 80 may also determine the amplitude of the cardiac pacing, cardioversion, or defibrillation pulses or other therapy waveforms.

Interval counters maintained by processor 80 may be reset upon sensing of R-waves and P-waves with detection channels of electrical sensing module 86. Processor 80 may also reset the interval counters upon the generation of pacing pulses by signal generator 98, and thereby control the basic timing of cardiac pacing functions, including CRT and ATP.

The value of the count present in the interval counters when reset by sensed R-waves and P-waves may be used by processor 80 to measure the durations of R-R intervals, P-P intervals, PR intervals and R-P intervals, which are measurements that may be stored in memory 82. Processor 80 may use the count in the interval counters to detect a tachyarrhythmia event, such as atrial or ventricular fibrillation or tachycardia. In some examples, a portion of memory 82 may be configured as a plurality of recirculating buffers, capable of holding series of measured intervals, which may be analyzed by processor 80 to determine whether the patient's heart 12 is presently exhibiting atrial or ventricular tachyarrhythmia.

In some examples, an arrhythmia detection method may include any suitable tachyarrhythmia detection algorithms. In one example, processor 80 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998. U.S. Pat. No. 5,545,186 to Olson et al. U.S. Pat. No. 5,755,736 to Gillberg et al. is incorporated herein by reference in their entireties. However, other tachyarrhythmia detection methodologies may also be employed by processor 80 in other examples.

Telemetry module 90 includes any suitable hardware, firmware, software or any combination thereof for communicating with another device, such as programmer 24 (FIG. 1). Under the control of processor 80, telemetry module 90 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 90, e.g., via an address/data bus. In some examples, telemetry module 90 may provide received data to processor 80 via a multiplexer.

In some examples, processor 80 may transmit atrial and ventricular heart signals (e.g., EGM signals) produced by atrial and ventricular sense amp circuits within electrical sensing module 86 and other physiological parameters sensors signals (e.g., blood pressure signal) to programmer 24. Programmer 24 may interrogate IMD 16 to receive the respective signals. Processor 80 may store the signals within memory 82, and retrieve stored signals from memory 82. Processor 80 may also generate and store marker codes indicative of different cardiac events that electrical sensing module 86 detects, such as atrial and ventricular depolarizations, and transmit the marker codes to programmer 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

The various components of IMD 16 may be coupled to power source 92, which may include a rechargeable or non-rechargeable battery and suitable power supply circuitry. A non-rechargeable battery may be selected to last for several years, while a rechargeable battery may be inductively charged from an external device, e.g., on a daily or weekly basis.

Further, in some examples of monitoring system 10, one or more aspects of sensing module 86 may be separate from IMD 16. That is, although all sensing functions attributed to IMD 16 using sensing module 86 are shown in FIG. 3 to be incorporated within or coupled to a housing of IMD 16 along with other components such as processor 80, in other examples, components for performing some or all of the functions attributed to sensing module 86 may be enclosed in a separate housing. In some examples, a stand-alone pressure sensing module, electrical activity sensing module and/or physiological parameter sensing module enclosed in one or more separate housing from the housing of IMD 16 may be mechanically coupled to IMD 16 or may be mechanically decoupled from IMD 16. For example, in some examples, a physiological parameter sensing module may be implanted within patient 14 at a separate location from IMD 16 and leads 18, 20, 22. Sensing modules outside the housing of IMD 16 may communicate with IMD 16 via a wired connection or via wireless communication techniques, such as RF telemetry.

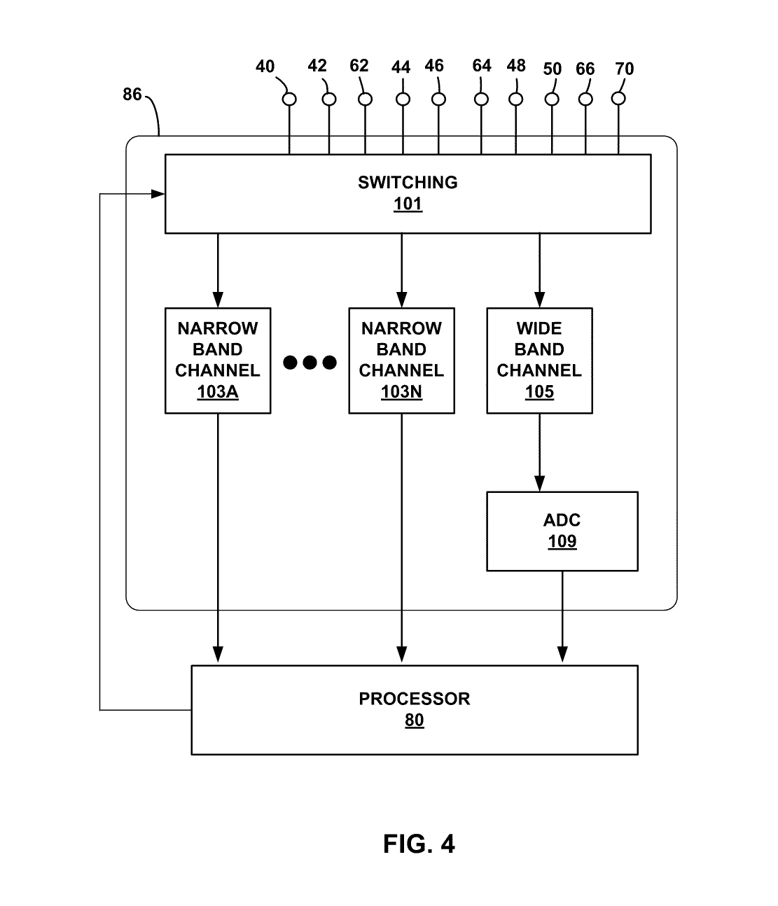

FIG. 4 is a functional block diagram illustrating an example sensing module 86 (FIG. 3). As shown in FIG. 4, electrical sensing module 86 includes multiple components including a switching module 101, narrow band channels 103A to 103N (collectively “narrow band channels 103”), wide band channel 105, and analog to digital converter (ADC) 109. Switching module 101 may, based on control signals from processor 80, control which of electrodes 40, 42, 44, 46, 48, 50, 62, 64, 66, and 70 is coupled to which of channels 103 and 105, at any given time.

Each of narrow band channels 103 may comprise a narrow band filtered sense-amplifier that compares the detected signal to a threshold. If the filtered and amplified signal is greater than the threshold, the narrow band channel indicates that a certain electrical heart event has occurred. Processor 80 then uses that detection in measuring frequencies of the detected events. Narrow band channels 103 may have distinct functions. For example, some various narrow band channels may be used to detect either atrial or ventricular events, e.g., atrial fibrillation.

In one example, at least one narrow band channel 103 may include an R-wave amplifier that receives signals from the sensing electrode configuration of electrodes 40 and 42, which are used for sensing and/or pacing in right ventricle 28 of heart 12. Another narrow band channel 103 may include another R-wave amplifier that receives signals from the sensing electrode configuration of electrodes 44 and 46, which are used for sensing and/or pacing proximate to left ventricle 32 of heart 12. In some examples, the R-wave amplifiers may take the form of an automatic gain controlled amplifier that provides an adjustable sensing threshold as a function of the measured R-wave amplitude of the heart rhythm.

In addition, in some examples, a narrow band channel 103 may include a P-wave amplifier that receives signals from electrodes 48 and 50, which are used for pacing and sensing in right atrium 26 of heart 12. In some examples, the P-wave amplifier may take the form of an automatic gain controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of sensing module 86 may be selectively coupled to housing electrode 70, or elongated electrodes 62, 64, or 66, with or instead of one or more of electrodes 40, 42, 44, 46, 48 or 50, e.g., for unipolar sensing of R-waves or P-waves in any of chambers 26, 28, or 32 of heart 12.

Wide band channel 105 may comprise an amplifier with a relatively wider pass band than the R-wave or P-wave amplifiers. Signals from the sensing electrode configuration that is selected for coupling to this wide-band amplifier may be converted to multi-bit digital signals by ADC 109. In some examples, processor 80 may store signals the digitized versions of signals from wide band channel 105 in memory 82 as EGMs. In some examples, the storage of such EGMs in memory 82 may be under the control of a direct memory access circuit.

In some examples, processor 80 may employ digital signal analysis techniques to characterize the digitized signals from wide band channel 105 to, for example detect and classify the patient's heart rhythm. Processor 80 may detect and classify the patient's heart rhythm by employing any of the numerous signal processing methodologies known in the art. Further, in some examples, processor 80 may analyze the morphology of the digitized signals from wide band channel 105 to distinguish between noise and cardiac depolarizations. Based on such morphological analysis, processor may detect a suspected non-physiological NST.

Based on the signals received from electrodes 40, 42, 44, 46, 48, 50, 62, 64, 66, and/or 70, sensing module 86 and/or processor 80 may detect the presence of a stroke risk factor. For example, sensing module 86 may utilize the P-wave amplifier to monitor the time interval between consecutive P-waves in heart 12, e.g., right atrium 26 of heart 12. Based on the time interval between consecutive sensed P-waves, sensing module 86 may detect the presence of atrial fibrillation. For example, if sensing module 86 and/or processor 80 determines that a certain percentage or amount of time intervals between P-waves over a particular time period are less than a threshold amount, sensing module 86 and/or processor 80 may detect the presence of atrial fibrillation in heart 14. Other suitable methodologies may be used to detect atrial fibrillation. In some examples, system 10 may characterize atrial fibrillation as being present as a stroke risk factor if heart 12 of patient 14 is concurrently experiencing atrial fibrillation or may be based on the detection of an atrial fibrillation burden exceeding a threshold. While examples of the disclosure are described with regard to atrial fibrillation, system 10 may detect any atrial tachyarrhythmia, which may include atrial fibrillation and atrial tachycardia, as a stroke risk factor based on cardiac electrical signals monitored by sensing module 86.

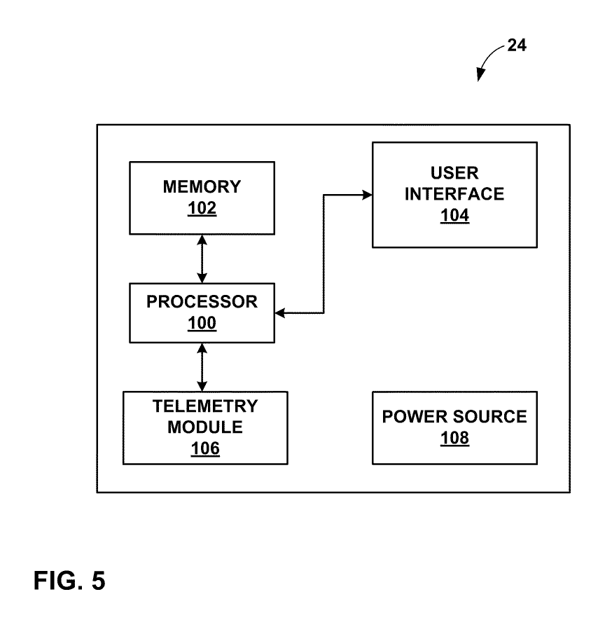

FIG. 5 is a functional block diagram of an example programmer 24. As shown in FIG. 5, programmer 24 includes processor 100, memory 102, user interface 104, telemetry module 106, and power source 108. Programmer 24 may be a dedicated hardware device with dedicated software for programming of IMD 16. Alternatively, programmer 24 may be an off-the-shelf computing device running an application that enables programmer 24 to program IMD 16.

A user such as a clinician may use programmer 24 to select therapy programs (e.g., sets of stimulation parameters), generate new therapy programs, modify therapy programs through individual or global adjustments or transmit the new programs to a medical device, such as IMD 16 (FIGS. 2 and 4). The user may also use programmer 24 to program or modify parameters related to the detection of stroke risk factors and/or generation of stroke risk score for patient 14, such as, for example, particular methodologies for detecting stroke risk factors via monitored physiological parameter information, algorithms for computing patient stroke risk scores, and/or threshold values to which stroke risk score may be are compared to determine whether or not to generate an alert to a user. In some examples, the user also may utilize programmer 24 to modify the frequency or length of stroke risk parameter detection intervals and/or the frequency at which a patient stroke risk score is computed. The user may interact with programmer 24 via user interface 104, which may include a display to present graphical user interface to a user, and a keypad or another mechanism for receiving input from a user.

The user also may use programmer 24 to retrieve data stored in memory 82 of IMD 16, such as, for example, physiological parameters sensed by sensors communicatively coupled to IMD 16. The physiological parameters may be used by programmer 24 to detect one or more patient stroke risk factors and/or to compute a patient stroke risk score as described in this disclosure. The user further may use programmer 24 to retrieve information regarding stroke risk factors detected for patient 14 and/or stroke risk score(s) stored in memory 82, if computed within IMD 16, or other measurements or information related to the monitoring the stroke risk of patient 14 via system 10. Hence, the detection of one or more stroke risk factors based on one or more monitored physiological parameter and/or generation of patient stroke risk score(s) may be performed within IMD 16 or within programmer 24.

Processor 100 can take the form one or more microprocessors, DSPs, ASICs, FPGAs, programmable logic circuitry, or the like, and the functions attributed to processor 102 herein may be embodied as hardware, firmware, software or any combination thereof. Memory 102 may store instructions that cause processor 100 to provide the functionality ascribed to programmer 24 herein, and information used by processor 100 to provide the functionality ascribed to programmer 24 herein.

Memory 102 may include any fixed or removable magnetic, optical, or electrical media, such as RAM, ROM, CD-ROM, hard or floppy magnetic disks, EEPROM, or the like. Memory 102 may also include a removable memory portion that may be used to provide memory updates or increases in memory capacities. A removable memory may also allow patient data to be easily transferred to another computing device, or to be removed before programmer 24 is used to program therapy for another patient. Memory 102 may also store information that controls therapy delivery by IMD 16, such as stimulation parameter values.

Programmer 24 may communicate wirelessly with IMD 16, such as using RF communication or proximal inductive interaction. This wireless communication is possible through the use of telemetry module 106, which may be coupled to an internal antenna or an external antenna. An external antenna that is coupled to programmer 24 may be placed over heart 12. Telemetry module 106 may be similar to telemetry module 90 of IMD 16 (FIGS. 3).

Telemetry module 106 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection.

Power source 108 delivers operating power to the components of programmer 24. Power source 108 may include a battery and a power generation circuit to produce the operating power. In some examples, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within programmer 24. In other examples, traditional batteries (e.g., nickel cadmium or lithium ion batteries) may be used. In addition, programmer 24 may be directly coupled to an alternating current outlet to power programmer 24. Power source 108 may include circuitry to monitor power remaining within a battery. In this manner, user interface 104 may provide a current battery level indicator or low battery level indicator when the battery needs to be replaced or recharged. In some cases, power source 108 may be capable of estimating the remaining time of operation using the current battery.

As described above, examples of the disclosure include systems, such as, stroke monitoring system 10, configured to monitor the stroke risk of a patient by monitoring one or more physiological parameters of the patient via an implantable medical device. Based on the one or more physiological factors monitored via the implantable medical device, the system may identify the presence of one or more stroke risk factors possessed by the patient. The system may evaluate the stroke risk of the patient based on the stroke risk factor(s) detected by the system by computing a stroke risk score that is reflective of the actual stroke risk of the patient. In some examples, the system may generate an indicator to alert a user of elevated and/or undesirable risk of stroke in the patient based on the stroke risk score.

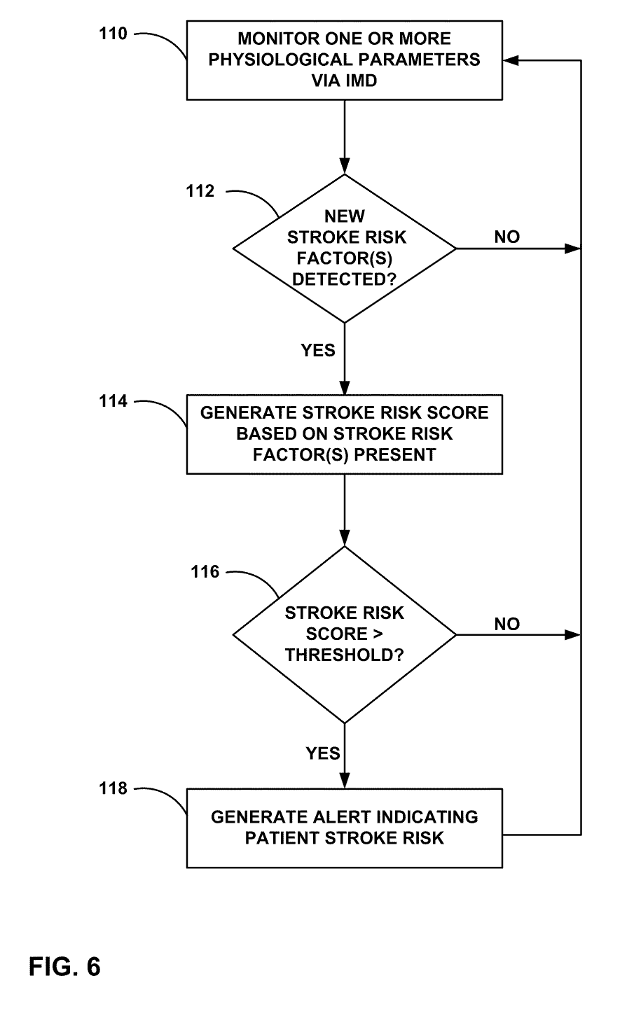

FIG. 6 is a flow diagram illustrating an example technique of monitoring the stroke risk of a patient via a stroke risk monitoring system. Although the technique shown may be performed via any suitable device or system including one or more implantable medical devices, the examples technique of FIG. 6 will be described with regard to stroke monitoring system 10 (FIG. 1) for purposes of illustration. As describe above, in addition to monitoring the stroke risk of patient 14, system 10 may be configured to deliver pacing, cardioversion, and/or defibrillation stimulation therapy generated via IMD 16 to heart 12 of patient 14. Additionally or alternatively, system 10 may be configured to deliver non-cardiac stimulation therapy to patient 14, e.g., neurostimulation therapy, or may be configured to monitor the stroke risk of patient 14 without delivering stimulation therapy to patient 14.

To illustrate one or more aspects of the example technique of FIG. 6, the example technique will be described with system 10 being configured to evaluate the stroke risk of patient 14 in a manner consistent with a modified CHADS2 scoring assessment. For example, for purposes of computing a stroke risk score, system 14 will generate a stroke risk score as a numerical value reflective of the overall patient stroke risk. In such an example, stroke risk factors used to compute the risk score include atrial fibrillation, congestive heart failure, hypertension, patient age greater than 75, diabetes, prior stroke or transient ischemic attack (TIA), with each factor being assigned a value of “1” in terms of the patient stroke risk score, except for prior stroke or TIA being assigned a value of “2.”

To monitor the stroke risk of patient 14, IMD 16 monitors one or more physiological parameters of patient 14 via sensing module 86 (110). For example, sensing module 86 may monitor pressure within heart 12 or other physiological location within patient 14 via pressure sensor 34, monitor the blood sugar level of patient 14 via physiological sensor 23, and monitor the electrical activity of heart 12 via one or more of electrodes 40, 42, 44, 46, 48, 50, 62, 64, 66, 70. Sensing module 86 monitor such physiological parameters of patient 14 on a periodic or continuous basis.

Based on the on the physiological parameters monitored via sensing module 86, sensing module 86, processor 80, or other processor may determine whether or not one or more stroke risk factors that influence the stroke risk score are present in patient 14 (112). For example, processor 80 or sensing module 86 may analyze the signals from pressure sensor 34 to determine whether or not hypertension is present, and may also analyze signals from physiological parameter sensor 23 to determine whether or not patient 14 has diabetes (e.g., when physiological parameter sensor 23 is configured to monitor the blood sugar level of patient 14). Similarly, processor 80 or sensing module 86 may also analyze signals sensed by one or more of electrodes 40, 42, 44, 46, 48, 50, 62, 64, 66, 70 indicating electrical activity of heart 12 to determine whether or not atrial fibrillation is present in patient 14. Processor 80 or sensing module 86 may also analyze signals sensed by one or more of electrodes 40, 42, 44, 46, 48, 50, 62, 64, 66, 70 indicating intrathoracic impedance or flow, or signals sensed by sensor 34 indicating pressure or flow, to determine whether or not congestive heart failure is present in patient 14. In this manner, system 10 detects the presence without directly receiving input from a user, but rather based on the physiological parameters monitored via sensing module 86 of IMD 16.

If system 10 detects that no new stroke risk factors are present in patient 14, sensing module 86 of IMD 16 continues to monitor the one or more physiological parameters of patient 14 without generating a stroke risk score or new stroke risk score if a stroke risk score exists for the patient based on previous identification of stroke risk factors. Conversely, if system 10 detects the presence of a new stroke risk factor in patient 14, system 10 generates a stroke risk score that is based on the one or more stroke risk factor present in patient 14 (114).

Processor 80 may compute the stroke risk score for patient 14 (114) by determining each stroke risk factor present in patient 14, each of which have a corresponding numerical value assigned, and then computing the aggregate of the stroke risk factor values of those stroke risk factors detected in patient via the monitored physiological parameters (e.g., hypertension, congestive heart failure, diabetes, atrial fibrillation) and/or those detected based on other information, e.g., information received from a user, such as, patient age, prior stroke or TIA.

As one example, system 10 may detect the presence of hypertension, diabetes, and atrial fibrillation in patient 14 via sensing module 86 (each of which are assigned a value of “1”), and also may detect the presence of past stroke in patient based on information received from a user via programmer 24 (which is assigned a value of “2”), with at least one of the stroke risk factors detected for patient 14 being new, e.g., not detected previously by system 10. In such an example, processor 80 may determine the stroke risk score of patient 14 as a value of “5” (i.e., 1+1+1+2), which is reflective of the overall stroke risk of patient indicated by the stroke risk factors detected in patient 14.

Upon determining the stroke risk score, processor 80 or processor 100 may compare the stroke risk score value to a threshold value (116). Based on the comparison, processor 80 or processor 100 determines whether or not to generate an indication indicating the stroke risk score of patient 14. If the computed risk score value is equal to or less than the threshold value, then processor 80 or processor 100 does not generate the indicator and sensing module 86 of IMD 16 continues to monitor the one or more physiological parameters of patient 14 to detect the presence of any new stroke risk factors.

Conversely, if the computed stroke risk score value is greater than the threshold value, then processor 80 or processor 100 may generate an indicator which indicates the patient stroke risk score (118). In some example, the indicator may be communicated to a user, e.g., via user interface 104 of programmer 24, to alert the user that system 10 has identified a stroke risk score that is greater than the threshold value. The indicator may indicate the actual stroke risk score value to the user and/or may provide some other indication that stroke risk monitoring system 10 has detected a risk score greater than the threshold value. In some examples, the indicator may alert patient 14 that system 10 has detected an elevated stroke risk for patient 14 and that a clinician should be consulted.

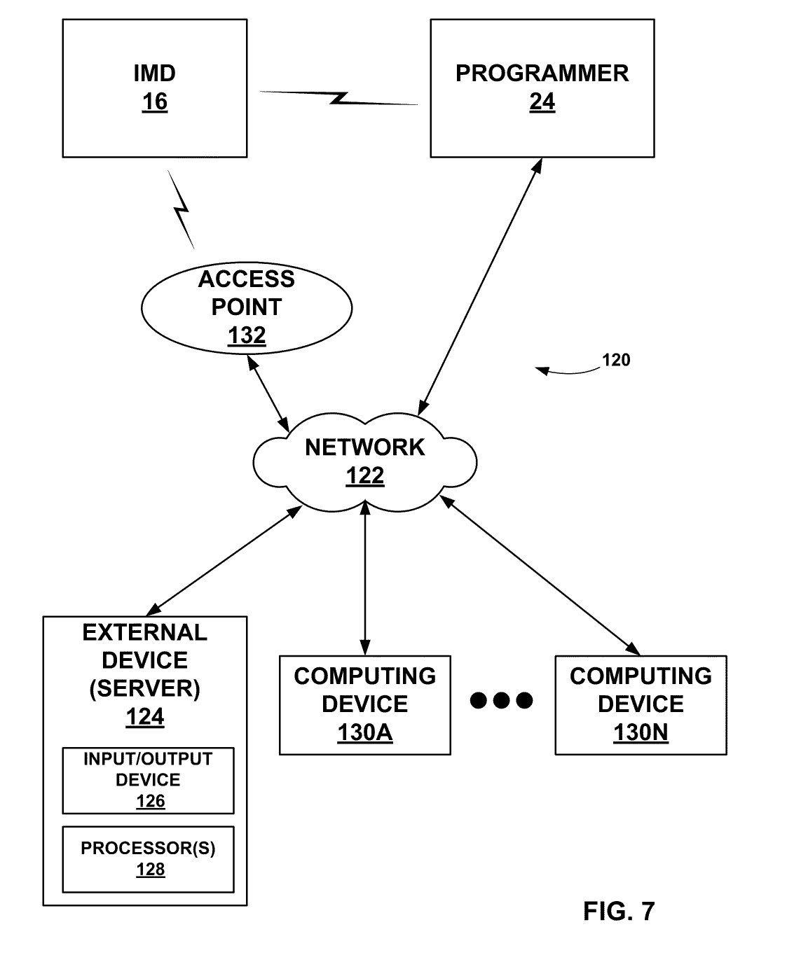

In some examples, upon detection of a stroke risk value that is greater than a threshold value, system 10 may contact a clinician via a remote communication system used to monitor information generated by system 10. One example of such a system may include the example system 120 shown in FIG. 7. Based on the communication received from system 10, the clinician may evaluate the proper response to the detected stroke risk score. For example, clinician may determine that patient 14 should be prescribed an anticoagulant or other treatment to treat the elevated stroke risk associated with the stroke computed by system 10. In some examples, the indicator may include information other than the stroke risk score of patient 14 to assist a clinician in evaluating the proper response to the detected stroke risk score, e.g., the indicator may include information regarding the particular stroke risk factors detected in patient 14 or identify any new stroke risk factor detected in patient 14 which was not present when any previous stroke risk score was computed by system 10.

Any suitable technique may be used to determine when system 10 generates an indicator indicating the stroke risk score of patient 14, e.g., to patient 14 and/or a clinician. In some examples, system 10 may generate such an indicator substantially any time a patient stroke risk score is computed by system 10 and/or a system 10 detects a change in the patient risk score. As in FIG. 6, a threshold value may be defined to determine when system 10 generates an indicator indicating the patient stroke score. The threshold value used to determine whether or not system 10 generates an indicator indicating the patient stroke risk score may be preprogrammed by a user, e.g., via programmer 24. The particular threshold value may be selected such that a relatively low stroke risk score generated by system 10 triggers an indicator, e.g., to alert patient 14 and/or clinician. In other examples, the threshold value may be selected such that a relatively high stroke risk score generated by system triggers an indicator, thereby notifying a user only when the stroke risk of patient 14 is relatively high. Multiple threshold values may be specified with different threshold values triggering different indicators by system 10, e.g., indicators indicating different strata of patient stroke risk levels. In some cases, an indicator may be generated by system 10 to alert a user to a reduction in the stroke risk of a patient. In such a case, based on such an alert, a clinician may determine whether or not to terminate or modify treatment currently being provided to patient 14, e.g., via prescription of anticoagulants, in a manner consistent with the reduced patient stroke risk.

In some examples, the threshold value used to trigger an indicator indicating the stroke risk score of patient 14 may be defined based on one or more previously generated stroke risk scores for patient 14. For example, the threshold value that triggers the generation of indicator by system 10 may be defined as the most recent stroke risk score value computed prior to the new stroke risk score value computed by system 10. In this manner, system 10 may only generate an indicator when the patient stroke risk score has increased. In some examples, such a protocol may be implemented by system 10 only after the computed stroke risk score is determined to be greater than some minimum value corresponding to an acceptable stroke risk score.

In some examples, system 10 may automatically or semi-automatically (e.g., upon clinician approval) modify one or more parameters of the stimulation therapy delivered from IMD 16 to patient 14 in response to the stroke risk score of a patient. For example, if the stroke risk of a patient associate with the stroke risk score computed by system 10 is relatively high, system 10 may modify the stimulation therapy delivered to patient 14 to be more aggressive in treating one or more patient conditions, e.g., those patient conditions which may be a precursor to stroke and/or those patient conditions that are also stroke risk factors. In one example, IMD 14 may modify the therapy delivered to patient 14 to more aggressively treat occurrences of atrial fibrillation in heart 12 of patient 14, which may be closely associated with stroke, upon receiving an indication that patient stroke risk score indicates a relatively high risk of stroke. In such a case, while providing stimulation therapy to treat atrial fibrillation of heart 12 may be outweighed by one or more undesirable side effects of the therapy when the stroke risk of patient 14 was relatively low, the provision of such stimulation therapy by IMD 14 to heart 12 may be deemed appropriate in view of the high stroke risk of patient 14 detected at that time per the generated stroke risk score.

System 10 may generate a stroke risk score for patient 14 on any suitable basis. In the example of FIG. 6, system 10 generates a stroke risk score for patient 14 upon detection of any new stroke risk factor. In some examples, system 10 may be configured to compute a stroke risk score for patient 14 on a substantially continuously basis, e.g., in real-time. In other examples, system 10 may be configured to compute the stroke risk score of patient 14 on a periodic basis. For example, a clinician may use programmer 24 to select the frequency at which system 10 computes the stroke risk score of a patient. In some examples, system 10 may be configured to update the stroke risk score of patient 14 on an hourly, daily, weekly, monthly or other suitable basis. In other examples, system 10 may compute a stroke risk score for patient 14 based on user input, e.g., based on a command from patient 14 and/or a clinician communicated to system 10 via programmer 24.

IMD 14 may monitor the physiological parameters used to detect even though system 10 is not actively computing stroke risk scores for patient 14 and/or detecting stroke risk factors. In some examples, IMD 14 may monitor one or more physiological parameters of patient and detect one or more stroke risk factors based on the monitored parameters to determine when one or more particular stroke risk factors that are defined as “activators” of the stroke risk score generation aspect of system 10. For example, system 10 may be configured to detect the presence of atrial fibrillation by on electrical activity of heart 12 monitored via sensing module 86. System 10 may actively detect other stroke risk factors during that time or system 10 may only analyze the monitored electrical activity to identify the presence of atrial fibrillation, at least for purposes of monitoring patient stroke risk. If system 10 fails to detect the presence of atrial fibrillation, then the stroke risk score generation aspect of system 10 may be inactive. However, once system 10 identifies the presence of atrial fibrillation in heart 12 of patient 14 based on the monitored electrical activity, system 10 may activate the stroke risk score generation aspect. When the stroke risk score generation aspect in active, system 10 may analyze monitored physiological parameter information to identify the presence of stroke risks other than that of atrial fibrillation and generate patient stroke risk scores when appropriate, e.g., upon identification of a new stroke risk factor in patient 14. Such an approach may be incorporated in cases in which specific stroke risk factors, e.g., atrial fibrillation correlate with relatively great risk of stroke, especially when present in patient 14 in combination with one or more other stroke risk factors.

IMD 14 may monitor any physiological parameters of patient 14 suitable to detect the presence of one or more stroke risk factors used to generate a stroke risk score. As described above, sensing module 86 may monitor blood pressure, electrical activity, e.g., cardiac electrical activity, blood sugar levels, and/or other physiological parameter that may be suitable for identifying the presence of one or more stroke risk factors. To detect the presence of stroke risk factors based on the monitored physiological parameters, processor 80 or processor 100 may analyze parameter information using any suitable methodology. In some examples, processor 80 or processor 100 may analyze the sensed parameter information to detect trends or occurrence which may indicate the presence of a particular stroke risk factor. In some examples, multiple physiological parameters may be monitored to identify the presence of a single risk factor in patient 12.

The stroke risk score generated by system 10 may take into account any suitable stroke risk factor which correlates to higher stroke risk for patient 14. Suitable stroke risk factors may include, but are not limited to, hypertension, congestive heart failure, diabetes mellitus, prior stroke or transient ischemic attack, atrial fibrillation, high blood cholesterol, obesity, sickle cell disease, and the like. The presence of one or more of such factor may be detected by monitoring one or more physiological parameters via IMD 16. As described above, system 10 may also detect the presence of one or more stroke risk factors (e.g., patient sex, age, prior stroke or TIA) without basing the detection on the one or more physiological parameters monitored by IMD 16.

In addition to monitoring one or more physiological parameters of patient 14 to detect the presence of one or more stroke risk factor in patient 14 via IMD 16, system 10 may detect the presence of patient risk factors based on other information. For example, the sex and/or age of patient 14 may be indicated to system 10 by a user, such as, patient 14 or a clinician, via programmer 24. In the case of patient age, the user may directly indicate the age of patient as presenting a stroke risk factor, or the user may indicate the age of the patient or date of birth, and system 10 may actively track the age of patient 14 to determine when the patient's age qualifies as a stroke risk factor, e.g., by tracking the age of patient 14 to detect when the age is over a benchmark age defining a stroke risk factor. In some examples, a user may indicate the presence of prior events, such as, prior stroke or TIA, which may be considered stroke risk factors, especially in cases in which IMD 14 was not implanted in patient 14 or actively detecting such factors in patient 14 at the time of the event. In some examples, a user may indicate the medication status of patient 14, e.g., whether or not patient 14 is regularly taking aspirin (which may decrease stroke risk). The stroke risk score generated by system 10 may be based on one or more stroke risk factors identified without monitoring of a physiological parameter, in combination with one or more stroke risk factor identified in view of the physiological parameter(s) monitored via IMD 14.

FIG. 7 is a block diagram illustrating an example system 120 that includes an external device, such as a server, and one or more computing devices that are coupled to IMD 16 and programmer 24 shown in FIG. 1 via a network. In some implementations, physiological signal data may be transmitted from IMD 16 to programmer 24 or another device and, in turn, to a server and/or client computers coupled to programmer 24 or the other device via a network. In this case, a remote server may compute a patient stroke risk score and/or the presence of one or more stroke risk factors based on information received from IMD 16 and/or programmer 24. Alternatively, information related to stroke risk score and/or stroke risk factors generated by IMD 16 or programmer 24 may be transmitted to such a remote server or client computer for processing, archival and/or viewing by a clinician or other caregiver.

In the example of FIG. 7, example system 120 includes an external device, such as a server 124, and one or more client computing devices 130A-130N, that are coupled to the IMD 16 and programmer 24 shown in FIG. 1 via a network 122. In this example, IMD 16 may use its telemetry module 90 to communicate with programmer 24 via a first wireless connection, and to communicate with an access point 132 via a second wireless connection. In the example of FIG. 7, access point 132, programmer 24, server 124, and computing devices 130A-130N are interconnected, and able to communicate with each other, through network 122.

In some cases, one or more of access point 132, programmer 24, server 124, and computing devices 130A-130N may be coupled to network 122 through one or more wireless connections. IMD 16, programmer 24, server 124, and computing devices 130A-130N may each comprise one or more processors, such as one or more microprocessors, DSPs, ASICs, FPGAs, programmable logic circuitry, or the like, that may perform various functions and operations, such as those described herein. For example, as illustrated in FIG. 7, server 124 may comprise one or more processors 128 and an input/output device 126, which need not be co-located.

Server 124 may, for example, implement any of the methods described herein for tracking the stroke risk for patient 14, including generation of the stroke risk score itself and any intermediate operations, such as determining the presence of one or more stroke risk factors based on monitored physiological parameter information, e.g., information related to pressure signals, cardiac signals, or other information. Server 124 also may provide a database or other memory for storing such information.

Access point 132 may comprise a device that connects to network 122 via any of a variety of connections, such as telephone dial-up, digital subscriber line (DSL), or cable modem connections. In other embodiments, access point 132 may be coupled to network 122 through different forms of connections, including wired or wireless connections. In some embodiments, access point 132 may be co-located with patient 14 and may comprise one or more programming units and/or computing devices (e.g., one or more monitoring units) that may perform various functions and operations described herein. For example, access point 132 may include a home-monitoring unit that is co-located with patient 14 and that may monitor the activity of IMD 16. In some embodiments, server 124 or one or more of the computing devices 130A-130N may perform any of the various functions or operations described herein.

Network 122 may comprise a local area network, wide area network, or global network, such as the Internet. In some cases, programmer 24 or server 124 may assemble one or more risk score values compute by system 10, stroke risk factors detected by system 10 or other data in web pages or other documents for viewing by trained professionals, such as clinicians, via viewing terminals associated with computing devices 130A-130N. System 132 may be implemented, in some aspects, with general network technology and functionality similar to that provided by the Medtronic CareLink® Network developed by Medtronic, Inc., of Minneapolis, Minn.

The techniques described in this disclosure, including those attributed to IMD 16 or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, or other devices. The term “processor” or “processing circuitry” may generally refer to any of the foregoing circuitry, alone or in combination with other circuitry, or any other equivalent circuitry.

Such hardware, software, or firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

Various examples have been described. These and other examples are within the scope of the following claims.

1. A method comprising:
monitoring at least one physiological parameter of a patient via an implantable medical device;
determining whether each of a plurality stroke risk factors are present based at least in part on the at least one physiological parameters monitored via the implantable medical device; and
generating a stroke risk score based on the stroke risk factors determined to be present, wherein the stroke risk score is reflective of the patient's risk of stroke.

2. The method of claim 1, wherein the plurality of stroke risk factors include one or more of atrial fibrillation, hypertension, congestive heart failure, patient age above threshold, diabetes, prior stroke and prior transient ischemic attack.

3. The method of claim 1, further comprising:
comparing the stroke risk score to a threshold value; and
generating an indicator indicative of the stroke risk score based on the comparison.

4. The method of claim 3, further comprising presenting the indicator to a user via a user interface.

5. The method of claim 1, further comprising adjusting the delivery of electrical stimulation therapy to the patient based at least in part on the stroke risk score.

6. The method of claim 1, wherein the implantable medical device comprises an implantable cardiac device configured to deliver electrical stimulation to a heart of the patient.

7. The method of claim 1, wherein the one or more physiological parameters includes at least one of electrical activity of a heart of the patient, blood pressure of the patient, blood flow of the patient, blood sugar level of the patient, and intrathoracic impedance of the patient.

8. The method of claim 1, wherein the stroke risk score comprises a numerical value.

9. The method of claim 8, wherein the plurality of stroke risk factors are each associated with a stroke risk value used to generate the stroke risk score.

10. The method of claim 1, further comprising detecting the presence of atrial fibrillation, wherein the stroke risk score is generated based on both the stroke risk factors determined to be present and detecting the presence of atrial fibrillation.

11. The method of claim 1, wherein the stroke risk score is based on the presence of the two or more stroke risk factors.

12. A stroke risk monitoring system comprising:
an implantable medical device including a sensing module configured to monitor at least one physiological parameter of a patient; and
a processor configured to determine whether each of a plurality of stroke risk factors are present based at least in part the at least one physiological parameter, and generate a stroke risk score based on the stroke risk factors determined to be present,
wherein the stroke risk score is reflective of the patient's risk of stroke.

13. The system of claim 12, wherein the plurality of stroke risk factors include one or more of atrial fibrillation, hypertension, congestive heart failure, patient age above threshold, diabetes, prior stroke and prior transient ischemic attack.

14. The system of claim 12, wherein the processor is configured to compare the stroke risk score to a threshold value; and generate an indicator indicative of the stroke risk score based on the comparison.

15. The system of claim 14, further comprising a user interface configured to present the indicator to a user.

16. The system of claim 12, wherein the processor is configured to adjust the delivery of electrical stimulation therapy to the patient based at least in part on the stroke risk score.

17. The system of claim 12, wherein the implantable medical device comprises an implantable cardiac device configured to deliver electrical stimulation to a heart of the patient.

18. The system of claim 12, wherein the one or more physiological parameters includes at least one of electrical activity of a heart of the patient, blood pressure of the patient, blood flow of the patient, blood sugar level of the patient, and intrathoracic impedance of the patient.

19. The system of claim 12, wherein the stroke risk score comprises a numerical value.

20. The system of claim 19, wherein the plurality of stroke risk factors are each associated with a stroke risk value used to generate the stroke risk score.

21. The system of claim 12, wherein the processor is configured to detect the presence of atrial fibrillation, and generate the stroke risk score based on both the stroke risk factors determined to be present and detection of the presence of atrial fibrillation.

22. The system of claim 12, wherein the implantable medical device includes the processor.

23. The system of claim 12, further comprising an external computing device including the processor.

24. The system of claim 12, wherein the stroke risk score is based on the presence of the two or more stroke risk factors.

25. A stroke risk monitoring system comprising:
means for monitoring at least one physiology parameter of a patient via an implantable medical device;
means for determining whether each of a plurality stroke risk factors are present based at least in part on the at least one physiological parameters monitored via the implantable medical device; and
means for generating a stroke risk score based on the stroke risk factors determined to be present, wherein the stroke risk score is reflective of the patient's risk of stroke.

26. A computer-readable storage medium comprising instructions that cause a processor to:
monitor at least one physiology parameter of a patient via an implantable medical device;
determine whether each of a plurality stroke risk factors are present based at least in part on the at least one physiological parameters monitored via the implantable medical device; and
generate a stroke risk score based on the stroke risk factors determined to be present, wherein the stroke risk score is reflective of the patient's risk of stroke.
